(19) United States
(12) Patent Application Publication
(10) Pub. No.: US 2021/0063339 A1
(43) Pub. Date: Mar. 4, 2021

(54) URINE TESTING STRIP AND URINE TESTING SYSTEM

(71) Applicants: FORMOSA PLASTICS CORPORATION, Kaohsiung City (TW); Chang Gung University, Tao-Yuan (TW)

(72) Inventors: Yu-Chia KUO, Kaohsiung City (TW); Ching-Yuan CHEN, Kaohsiung City (TW); Po-Ming YANG, Kaohsiung City (TW); Shun-Wei SHIH, Kaohsiung City (TW); Cho-Wen HUANG, Kaohsiung City (TW); Jen-Chieh CHIEN, Kaohsiung City (TW); Wei-San CHANG, Kaohsiung City (TW); Mu-Yi HUA, Taoyuan City (TW); Ching-Fu CHEN, Kaohsiung City (TW); Yu-Cheng CHEN, Kaohsiung City (TW); Wan-Tun HUNG, Kaohsiung City (TW); Chin-Lien HUANG, Kaohsiung City (TW)

(21) Appl. No.: 17/002,196

(22) Filed: Aug. 25, 2020

(30) Foreign Application Priority Data
Aug. 26, 2019 (TW) 108130495

(51) Int. Cl. G01N 27/327; G01N 27/30; G01N 27/49

(52) U.S. Cl. CPC G01N 27/327; G01N 27/49; G01N 27/301; G01N 27/307

(57) ABSTRACT

A urine testing strip and a urine testing system are provided. The urine testing system includes the urine test strip and a detection device. The urine test strip includes a working electrode, a counter electrode, a reference electrode, a trigger circuit and a protection layer. The protection layer covers the working electrode, the counter electrode and the reference electrode, and portions of the working electrode, the counter electrode and the reference electrode are exposed through an opening of the protection layer. The detection device includes a test strip receiving module and a wireless transmission module. The test strip receiving module is used to receive the test strip and to provide electric power to the test strip for obtaining a detection result. The wireless transmission module is used to transmit the detection result to a mobile computer device.

### RELATED APPLICATIONS

This application claims priority to Taiwan Application Serial Number 108130495, filed on Aug. 26, 2019, which is herein incorporated by reference.

### BACKGROUND

### Field of Invention

The present invention relates to a urine testing strip and a urine testing system.

### Description of Related Art

In modern life, people may go to a medical center for a regular health check with a purpose of finding diseases at an early age and increasing the opportunity of curing the diseases. However, it is difficult for people to go to a medical center for a regular health check due to the restrictions of time, traffic, job, etc. Therefore, the people in the business develop various cheap measurement meters easy to carry, such as a sphygmomanometer, a blood glucose meter, a body fat meter and a urine testing meter, for people to conduct the health check by themselves.

### SUMMARY

Embodiments of the present invention provide a urine testing strip and a urine testing system for people to conveniently conduct a rapid urine screening by themselves.

In accordance with some embodiments of the present invention, the urine testing strip includes a substrate, a working electrode, a counter electrode, a reference electrode, a trigger circuit and a protection layer. The working electrode is disposed on a surface of the substrate. The counter electrode is disposed on the surface of the substrate, in which the counter electrode and the working electrode are configured to provide a voltage difference to carry out an electrochemical reaction on a urine sample. The reference electrode is disposed on the surface of the substrate, in which the reference electrode is configured to provide a fixed reference voltage. The trigger circuit is disposed on the substrate. The protection layer covers the working electrode, the counter electrode and the reference electrode, in which the protection layer has an opening exposing a portion of the working electrode, a portion of the counter electrode and a portion of the reference electrode.

In some embodiments, the trigger circuit includes a U-shape conductive line.

In some embodiments, the U-shape conductive line includes two ends and a bended portion, the bended portion is covered by the protection layer, and the two ends are not covered by the protection layer.

In some embodiments, the working electrode includes a conductor and a sensing film disposed on the conductor, and the sensing film includes polyimide derivatives having heterocyclic aromatic groups.

In some embodiments, the heterocyclic aromatic groups include benzimidazole or benzoxazole.

In accordance with some embodiments of the present invention, the urine testing system includes a urine testing strip and a detection device. The urine testing strip is configured to receive a urine sample, in which the urine testing strip includes a substrate, a working electrode, a counter electrode, a reference electrode, a trigger circuit and a protection layer. The working electrode is disposed on a surface of the substrate. The counter electrode is disposed on the surface of the substrate, in which the counter electrode and the working electrode are configured to provide a voltage difference to carry out an electrochemical reaction on the urine sample. The reference electrode is disposed on the surface of the substrate, in which the reference electrode is configured to provide a fixed reference voltage. The trigger circuit is disposed on the substrate. The protection layer covers the working electrode, the counter electrode and the reference electrode, in which the protection layer has an opening exposing a portion of the working electrode, a portion of the counter electrode and a portion of the reference electrode. The detection device is configured to receive the urine testing strip and to perform a detection operation on the urine sample through the urine testing strip, in which the detection device includes a test strip receiving module and a wireless transmission module. The test strip receiving module is configured to receive the test strip and to trigger the detection device though the trigger circuit, thereby providing electric power to the test strip for obtaining a detection result. The wireless transmission module is configured to receive the detection result and to transmit the detection result to a mobile computer device.

In some embodiments, the test strip receiving module includes an open-circuit circuitry, the open-circuit circuitry has two ends, and the open-circuit circuitry is turned on when two ends of the trigger circuit contact the ends of the open-circuit circuitry, thereby triggering the detection device to provide electric power to the test strip.

In some embodiments, when the detection device to is triggered to provide electric power to the test strip, the detection device applies voltages to the working electrode, the counter electrode and the reference electrode.

In some embodiments, the working electrode includes a conductor and a sensing film disposed on the conductor, and the sensing film includes polyimide derivatives having heterocyclic aromatic groups.

In some embodiments, the mobile computer device is a smart phone.

### BRIEF DESCRIPTION OF THE DRAWINGS

The invention can be more fully understood by reading the following detailed description of the embodiment, with reference made to the accompanying drawings as follows.

FIG. 1a and FIG. 1b are schematic diagrams showing a urine testing strip in accordance with embodiments of the present invention.

FIG. 2a and FIG. 2b are schematic diagrams showing a urine testing strip in accordance with another embodiment of the present invention.

FIG. 3 is a functional block diagram of a detection device 300 of a urine testing system in accordance with an embodiment of the present invention.

### DETAILED DESCRIPTION

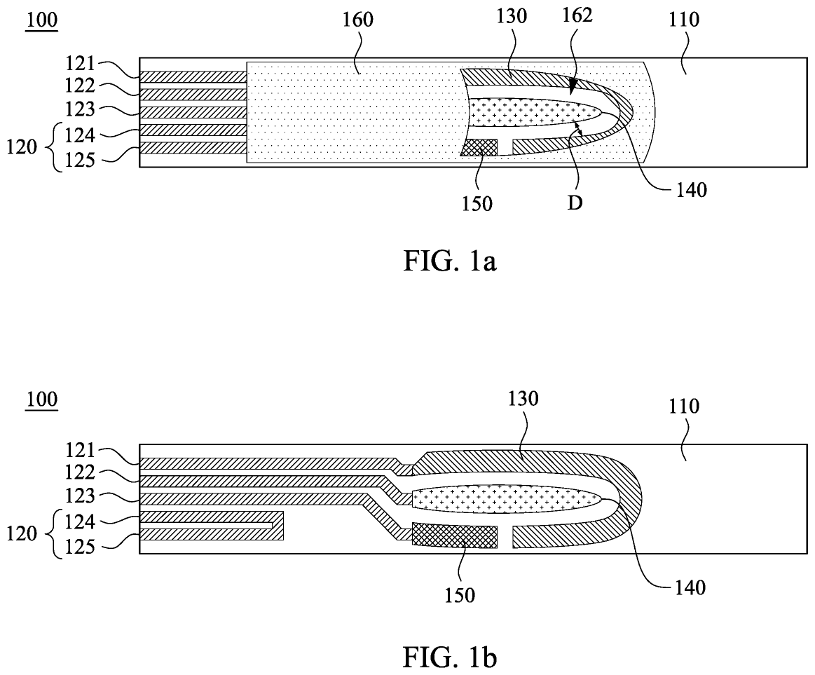

Referring to FIG. 1a and FIG. 1b, FIG. 1a and FIG. 1b are schematic diagrams showing a urine testing strip 100, in which a protection layer 160 is ignored (not shown) in FIG. 1b. The urine testing strip 100 is configured to receive a urine sample and to perform a detection operation on the urine sample. The urine testing strip 100 includes a substrate 110, conductive lines 121-125, a counter electrode 130, a working electrode 140, a reference electrode 150 and a protection layer 160. The counter electrode 130 and the working electrode 140 are configured to provide a voltage difference to carry out an electrochemical reaction on the urine sample, for example a reduction-oxidation reaction. In this embodiment, the working electrode 140 includes a conductor and a sensing film disposed on the conductor. In one embodiment, the conductor can be made of gold, platinum, copper or conductive carbon material, and the conductor is coated with the sensing film. However, embodiments of the present invention are not limited thereto.

The sensing film includes polyimide derivatives having heterocyclic aromatic groups, and a repeating unit thereof is shown below:

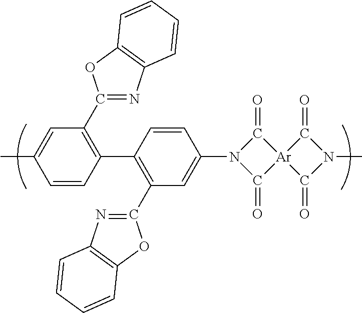

where Ar is a derivative of dianhydride compound such as pyromellitic dianhydride (PMDA), 3,3′,4,4′-biphenyltetracarboxylic dianhydride (BPDA), 4,4′-oxydiphthalic anhydride (ODPA) and 1,4,5,8-naphthalenetetracarboxylic dianhydride (NTDA).

Therefore, in this embodiment, the working electrode has a sensitivity to a detected target (i.e., Hydrogen peroxide), and thus the concentration of the detected target can be obtained through measuring the current produced from a reduction-oxidation reaction between the working electrode and the detected target and through a concentration calculation based on the measured current value. In other embodiments of the present invention, the heterocyclic aromatic groups may include benzimidazole or benzoxazole. To obtain better performance of measuring the current, a thickness of the sensing film is ranged from 5 micrometers (μm) to 15 micrometers. In one embodiment, a thickness of the sensing film is 10 micrometers.

The counter electrode 130 is disposed surrounding the working electrode 140, and there is a gap D between the counter electrode 130 and the working electrode 140. In this embodiment, the electrode gap D is ranged from 10 millimeter (mm) to 40 millimeter, and the material of the counter electrode 130 is gold. However, embodiments of the present invention are not limited thereto. The reference electrode 150 is disposed adjacent to the working electrode 140 to provide a fixed reference voltage, in which there is a gap between the reference electrode 150 and the working electrode 140. In this embodiment, the material of the reference electrode 150 may include but is not limited to a silver/silver chloride (Ag/AgCl) electrode, a silver electrode, a mercury electrode or other suitable material. In addition, the working electrode 140 has an area A1 the counter electrode 130 has an area A2; the reference electrode 150 has an area A3, in which an area ratio A1:A2:A3 is ranged from 3:3:0.5 to 1:1:1.

The protection layer 160 covers the conductive lines 121-125, the counter electrode 130, the working electrode 140 and the reference electrode 150 to protect the substrate 110, the conductive lines 121-125, the counter electrode 130, the working electrode 140 and the reference electrode 150, as shown in FIG. 1a. The protection layer 160 in this embodiment has an opening 162 exposing a portion of the counter electrode 130, a portion of the working electrode 140 and a portion of the reference electrode 150. Therefore, the urine testing strip 100 may receive the urine sample through the opening 162 and perform the detection operation on the urine sample in the opening 162. Ends of the conductive lines 121-125 close to edges of the substrate 110 are not covered by the protection layer 160. Therefore, after the urine testing strip 100 is plugged into a detection device, the ends of the conductive lines 121-125 directly contact corresponding ends in the detection device, thereby achieving electric connection between the urine testing strip 100 and the detection device.

Specifically, the conductive lines 124 and 125 are electrically connected to each other to form a trigger circuit 120. When the urine testing strip 100 is plugged into the detection device, the trigger circuit 120 may trigger the detection device to provide electric power to the urine testing strip 100. When the detection device provides electric power to the urine testing strip 100, the detection device may apply voltages to the counter electrode 130, the working electrode 140 and the reference electrode 150 to enable the urine testing strip 100 to perform the detection operation on the urine sample. In this embodiment, the conductive lines 124 and 125 are electrically connected to each other to form a U-shape conductive line. A bended portion of the U-shape conductive line is covered by the protection layer 160, and two ends of the U-shape conductive line are not covered by the protection layer 160 to provide electric connection of the trigger circuit 120.

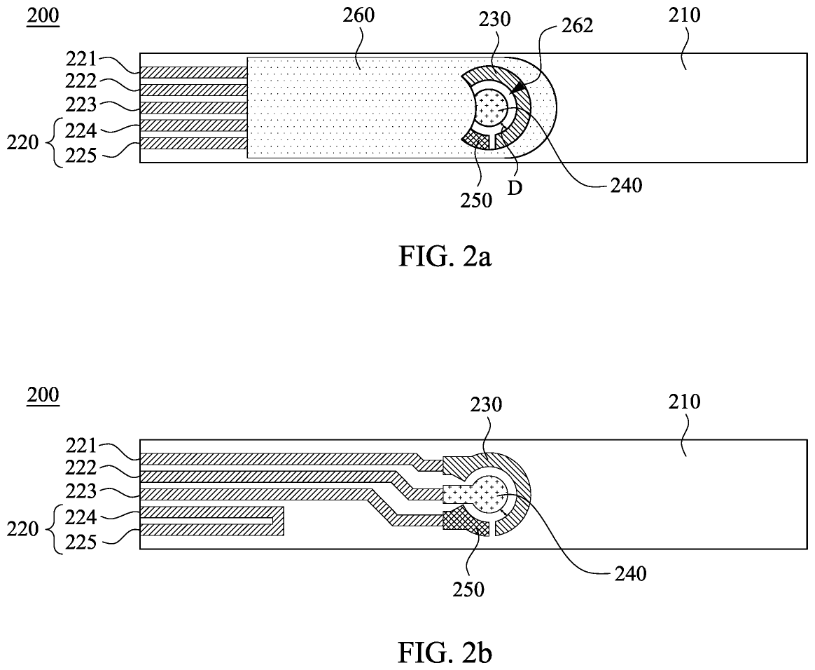

Referring to FIG. 2a and FIG. 2b, FIG. 2a and FIG. 2b are schematic diagrams showing a urine testing strip 200 in accordance with another embodiment of the present invention, in which a protection layer 260 is ignored (not shown) in FIG. 2b. The urine testing strip 200 includes a substrate 210, conductive lines 221-225, a counter electrode 230, a working electrode 240, a reference electrode 250 and a protection layer 260, in which the counter electrode 230 and the working electrode 240 are configured to provide a voltage difference to carry out an electrochemical reaction on a urine sample.

The counter electrode 230, the working electrode 240, the reference electrode 250 and the protection layer 260 are similar to the counter electrode 130, the working electrode 140, the reference electrode 150 and the protection layer 160 of the above embodiment, but the difference is in that the shapes of the counter electrode 230, the working electrode 240, the reference electrode 250 and the protection layer 260 are different from the shapes of the counter electrode 130, the working electrode 140, the reference electrode 150 and the protection layer 160. As shown in FIG. 2a, an opening of the protection layer 260 exposes a portion of the working electrode 240, and the portion of the working electrode 240 is circular. The shapes of the counter electrode 230 and the reference electrode 250 match the circular portion of the working electrode 240, and the counter electrode 230 and the reference electrode 250 are disposed surrounding the circular portion of the working electrode 240.

In addition, the urine testing strip 200 of this embodiment also includes a trigger circuit 220, and the trigger circuit 220 is configured to trigger the detection device to provide electric power. Because the structure of the trigger circuit 220 is similar to the structure of the trigger circuit 120, the structure of the trigger circuit 220 is not repeated herein.

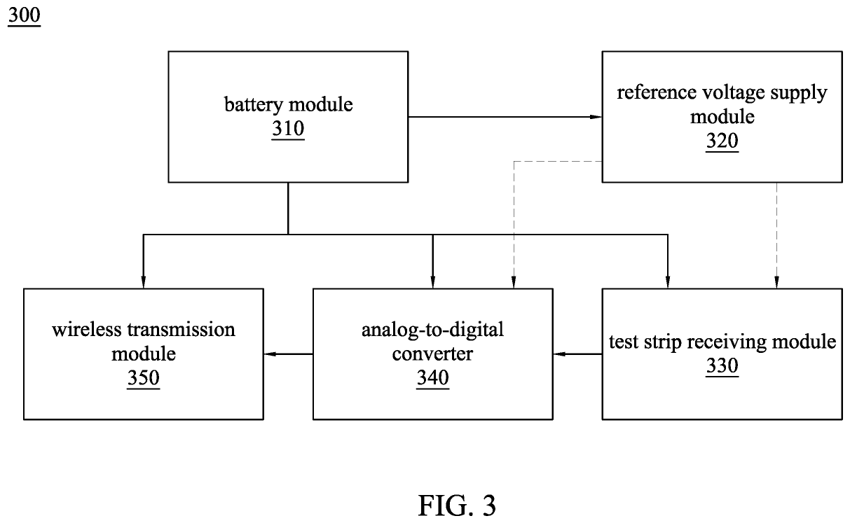

Referring to FIG. 3, FIG. 3 is a functional block diagram of a detection device 300 of a urine testing system in accordance with an embodiment of the present invention. The detection device 300 includes a battery module 310, a reference voltage supply module 320, a test strip receiving module 330, an analog-to-digital converter 340 and a wireless transmission module 350. The battery module 310 is configured to provide electric power to the reference voltage supply module 320, the test strip receiving module 330, the analog-to-digital converter 340 and the wireless transmission module 350. The reference voltage supply module 320 is configured to provide a reference voltage required by the test strip receiving module 330 and the analog-to-digital converter 340.

The test strip receiving module 330 is configured to receive the urine testing strip 100/200 to provide electric power to the urine testing strip 100/200 to enable the urine testing strip 100/200 to perform the detection operation on the urine sample and then obtain a detection result (i.e., the current value). In one embodiment, the test strip receiving module 330 has an open-circuit circuitry (not shown) having two ends. When the two ends of the trigger circuit 120/220 contact the two ends of the open-circuit circuitry, the open-circuit circuitry is turned on (short-circuited) to trigger the detection device 300 to provide electric power to the urine testing strip 100/200. After receiving the electric power, the urine testing strip 100/200 may perform the detection operation on the urine sample, and the test strip receiving module 330 may measure the current produced from the reduction-oxidation reaction between the working electrode and Hydrogen peroxide to obtain a value of the produced current. In this embodiment, the test strip receiving module 330 is an analog from end (AFE), but embodiments of the present invention are not limited thereto.

The analog-to-digital converter 340 is electrically connected to the test strip receiving module 330 to convert the detection result transmitted by the test strip receiving module 330 from an analog signal to a digital signal. The wireless transmission module 350 is electrically connected to the analog-to-digital converter 340 to transmit the detection result to a mobile computer device, for example the user's smart phone. After receiving the detection result, the smart phone may show the detection result on the screen thereof, and thus the user can understand the present state of health. In addition, the smart phone may transmit the detection result to a backstage (for example, a cloud database) to store the detection result in the backstage for convenience of inquiring the detection result at any time.

It can be understood from the above descriptions that the embodiments of the present invention provide a urine testing system including the urine testing strip 100/200 and the detection device 300. Because of the reduction-oxidation reaction generated between Hydrogen peroxide and the working electrode 140/240 of the urine testing strip 100/200, the urine testing system can measure the current produced from the reduction-oxidation reaction to obtain the concentration of the Hydrogen peroxide (i.e., the detection result). Thereafter, the detection result can be transmitted to the use's smart phone, and thus the user can understand the present state of health through the detection result shown on the smart phone. Therefore, the urine testing system of the embodiments of the present invention can achieve rapid urine screening.

Although the present invention has been described in considerable detail with reference to certain embodiments thereof, other embodiments are possible. Therefore, the spirit and scope of the appended claims should not be limited to the description of the embodiments contained herein. It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A urine testing strip, comprising:
a substrate;
a working electrode disposed on a surface of the substrate;
a counter electrode disposed on the surface of the substrate, wherein the counter electrode and the working electrode are configured to provide a voltage difference to carry out an electrochemical reaction on a urine sample;
a reference electrode disposed on the surface of the substrate, wherein the reference electrode is configured to provide a fixed reference voltage;
a trigger circuit disposed on the substrate; and
a protection layer covering the working electrode, the counter electrode and the reference electrode, wherein the protection layer has an opening exposing a portion of the working electrode, a portion of the counter electrode and a portion of the reference electrode.

2. The urine testing strip of claim 1, wherein the trigger circuit comprises a U-shape conductive line.

3. The urine testing strip of claim 2, wherein the U-shape conductive line comprises two ends and a bended portion, the bended portion is covered by the protection layer, and the two ends are not covered by the protection layer.

4. The urine testing strip of claim 1, wherein the working electrode comprises a conductor and a sensing film disposed on the conductor, and the sensing film comprises polyimide derivatives having heterocyclic aromatic groups.

5. The urine testing strip of claim 4, wherein the heterocyclic aromatic groups comprise benzimidazole or benzoxazole.

6. A urine testing system, comprising:
a urine testing strip configured to receive a urine sample, wherein the urine testing strip comprises:
a substrate;
a working electrode disposed on a surface of the substrate;
a counter electrode disposed on the surface of the substrate, wherein the counter electrode and the working electrode are configured to provide a voltage difference to carry out an electrochemical reaction on the urine sample;
a reference electrode disposed on the surface of the substrate, wherein the reference electrode is configured to provide a fixed reference voltage;
a trigger circuit disposed on the substrate; and
a protection layer covering the working electrode, the counter electrode and the reference electrode, wherein the protection layer has an opening exposing a portion of the working electrode, a portion of the counter electrode and a portion of the reference electrode; and
a detection device configured to receive the urine testing strip and to perform a detection operation on the urine sample through the urine testing strip, wherein the detection device comprises:
a test strip receiving module configured to receive the test strip and to trigger the detection device though the trigger circuit, thereby providing electric power to the test strip for obtaining a detection result; and
a wireless transmission module configured to receive the detection result and to transmit the detection result to a mobile computer device.

7. The urine testing system of claim 6, wherein the test strip receiving module comprises an open-circuit circuitry, the open-circuit circuitry has two ends, and the open-circuit circuitry is turned on when two ends of the trigger circuit contact the ends of the open-circuit circuitry, thereby triggering the detection device to provide electric power to the test strip.

8. The urine testing system of claim 7, wherein when the detection device to is triggered to provide electric power to the test strip, the detection device applies voltages to the working electrode, the counter electrode and the reference electrode.

9. The urine testing system of claim 6, wherein the working electrode comprises a conductor and a sensing film disposed on the conductor, and the sensing film comprises polyimide derivatives having heterocyclic aromatic groups.

10. The urine testing system of claim 6, wherein the mobile computer device is a smart phone.
